(19) United States
(12) Patent Application Publication
(10) Pub. No.: US 2009/0074721 A1
(43) Pub. Date: Mar. 19, 2009

(54) METHODS FOR TREATING VIRAL INFECTION WITH ORAL OR INJECTIBEL DRUG SOLUTION

(75) Inventors: Jong Joseph Kim, Harleyville, PA (US); Rajinder Matharu, Bloomington, IN (US)

Correspondence Address: Pepper Hamilton LLP, 400 Berwyn Park, 899 Cassatt Road, Berwyn, PA, 19312-1183, US

(73) Assignee: VGX PHARMACEUTICALS, INC., Blue Bell, PA (US)

(21) Appl. No.: 11/916,628

(22) PCT Filed: Jun. 6, 2006
(86) PCT No.: PCT/US06/21923

§ 371 Date: Oct. 9, 2008

Related U.S. Application Data
(60) Provisional application No. 60/687,813, filed on Jun. 6, 2005.

(51) Int. Cl. A61K 31/56; A61K 31/7088; A61K 31/704; A61K 38/21; A61P 31/12

(52) U.S. Cl. 424/85.7; 514/177; 514/178; 514/172; 514/26

(57) ABSTRACT

Pharmaceutical composition comprising compounds and/or compositions useful to inhibit viral replication are disclosed.

### CROSS-REFERENCE TO RELATED APPLICATION

The present application claims priority to U.S. Provisional Application No. 60/687,813, filed Jun. 6, 2005, which is hereby incorporated by reference in its entirety.

### FIELD OF THE INVENTION

The present invention relates to pharmaceutical compositions which includes a novel method to dissolve GR antagonist drugs, including Mifepristone, capable of being delivered in oral or injectible route to treat viral infection in individuals who are exposed to viruses.

### BACKGROUND OF THE INVENTION

HIV is a lentivirus whose genome contains only about 9-11 kb of genetic material and less than 10 open reading frames. HIV possesses a collection of small, positive strand open reading frames which encode 1-2 exon genes whose protein products regulate various aspects of the virus' life cycle. Some of these genes are genetic transactivating factors which are necessary for virus replication in all permissive cell types.

The progression from HIV infection to AIDS is in large part determined by the effects of HIV on the cells that it infects, including CD4⁺ T lymphocytes and macrophages. Cell activation, differentiation and proliferation in turn regulate HIV infection and replication in T cells and macrophages. Gallo, R. C. et al. (1984) Science 224:500; Levy, J. A. et al., (1984) Science 225:840; Zack, J. A. et al. (1988) Science 240:1026; Griffin, G. E. et al., (1988) Nature 339:70; Valentin, A. et al. (1991) J. AIDS 4:751; Rich, E. A. et al., (1992) J. Clin. Invest. 89:176; and Schuitemaker, H. et al. (1992) J. Virol. 66:1354. Cell division per se may not be required since HIV and other lentiviruses can proliferate in nonproliferating, terminally differentiated macrophages and growth-arrested T lymphocytes. Rose, R. M. et al. (1986) Am. Rev. Respir. Dis. 143:850; Salahuddin, S. Z. et al. (1986) Blood 68:281; and Li, G. et al. (1993) J. Virol. 67:3969. HIV infection of myeloid cell lines can result in a more differentiated phenotype and increase the expression of factors such as NF-KB which are necessary for HIV replication. Roulston, A. et al. (1992) J. Exp. Med. 175:751; and Chantal Petit, A. J. et al. (1987) J. Clin. Invest. 79:1883.

Since the demonstration in 1987 that the small open reading frame within HIV-1 designated R encodes a 15 KD protein (Wong-Staal, F., et al., (1987) AIDS Res. Hum. Retroviruses 3:33-39), there has been a growing body of literature regarding the function of the viral protein R (Vpr). The ability of lentiviruses, including HIV, to replicate in nonproliferating cells, particularly in macrophages, is believed to be unique among retroviruses. It is significant that several lentiviruses contain a vpr-like gene. Myers, G. et al. (1992) AIDS Res. Hum. Retrovir. 8:373. The vpr open reading frame is conserved within all genomes of HIV-1 and HIV-2 and within all pathogenic isolates of simian immunodeficiency virus (SIV) genomes. The evolutionary requirement for economy in design is deemed to require that the presence of vpr in the mV genome is related to a specific and non-dispensable function in the viral life cycle.

It has been reported that mutations in the vpr gene result in a decrease in the replication and cytopathogenicity of HIV-1, HIV-2, and SIV in primary CD4⁺ T lymphocytes and transformed T cell lines. See, e.g., Ogawa, K., et al., (1989) J. Virol. 63:4110-4114; Shibata, R., et al. (1990a) J. Med. Primatol. 19:217-225; Shibata, R., et al. (1990b) J. Virol. 64:742-747 and Westervelt, P. et al. (1992) J. Virol. 66:3925, although others have reported that mutated vpr gene had no effect on replication (Dedera, D., et al. (1989) Virol. 63:3205-3208). Importantly, HIV-2 mutated for vpr has been reported unable to infect primary monocyte/macrophages (Hattori, N., et al. (1990) Proc. Natl. Acad. Sci. USA 87:8080-8084). Further, viral replication in macrophages may be almost completely inhibited by antisense ribonucleotides targeting the vpr open reading frame. This, together with the induction of rhabdomyosarcoma cellular differentiation, are deemed to dictate a crucial function for Vpr in HIV pathogenesis.

The Vpr protein is the only HIV-1 regulatory gene product which has been shown to be incorporated into virions. This would normally suggest a structural role for Vpr, but since vpr deleted viruses are able to produce normal virions, this is deemed to be further evidence of a regulatory role for this molecule. The presence of Vpr in virions has been associated with increased replication kinetics in T lymphocytes, and with the ability of HIV to establish productive infection in monocytes and macrophages. The presence of Vpr protein in viral particles means an early function for Vpr during the infection process, following virus penetration and uncoating. This role is considered to involve Vpr interaction with cellular regulatory mechanisms resulting in an increase in cell permissiveness to sustain viral replication processes. See, e.g., Cohen, E. A., et al. 1990a J. Virol. 64:3097-3099; Yu, X. F., et al. (1990) J. Virol. 64:5688-5693; and, Yuan, X., et al., (1990) AIDS Res. Hum. Retroviruses 6:1265-1271.

U.S. Pat. No. 5,874,225, which is incorporated herein by reference, discloses several activities and characteristics of Vpr including its ability to inhibit cellular proliferation and its ability to associate with protein product encoded by the gag gene. Vpr action can involve the upregulation of cellular elements which enhance viral gene expression, or the downmodulation of cellular inhibitory pathways affecting such viral processes. Such cellular disregulation is consistent with the observation that Vpr is sufficient for the differentiation and cessation in cellular proliferation of rhabdomyosarcoma and osteosarcoma cell lines (Levy, D. N. et al. (1993) Cell 72:541). The ability of a virally associated protein such as Vpr to reinitiate an arrested developmental program is clearly based upon its interaction with other cellular proteins, and since Vpr protein originates within viral particles, it is considered that Vpr must, accordingly, play a role in establishing productive infection.

U.S. Pat. No. 5,780,238, which is incorporated herein by reference, describes the isolation of an approximately 41 KD Vpr cytosolic binding or interacting protein, which has been designated hereafter as Rip-1. As used herein, the term “Rip-1” is meant to refer to the human protein that has an apparent molecular weight of between 40-43 KD, that occurs in the cytoplasm of human cells, that binds to Vpr and that is transported from the cytoplasm to the nucleus when bound to Vpr, either alone or in association with a steroid receptor.

Rip-1 may be co-localized with the T-cell and B-cell transcription factor NfκB. Vpr and Rip-1 coelute in an immunoaffinity system, and can be specifically crosslinked to a 58 KD complex. Using peptide and antibody competition, the site of their interaction has been resolved to amino acids 38 to 60 on the Vpr amino acid sequence. Rip-1 has been detected in various cell lines. Rip-1 selectively translocates from the cytosol to the nucleus upon exposure of the cell to Vpr either in a soluble form, or through infection with wild type virus, but not in response to PMA, suggesting a coupling in their regulatory functions. Consequently, the present invention involves the discovery that Rip-1 may be partially responsible for mediating Vpr activity in the human host cell.

U.S. Pat. No. 5,639,598, which is incorporated herein by reference, refers to the discovery that HIV Vpr protein forms a complex with proteins, including Rip-1, in human cells that are in association with, i.e., as a part of or functionally combined with, one or more steroid receptors, especially the glucocorticoid receptor (GR). Inhibitory or antagonist compounds which bind to, or otherwise wholly or partially preclude the formation of a complex involving Vpr and steroid receptors, especially a GR-type receptor, or potentially other components, or one or more steroid receptors alone, prevent or interfere with HIV replication.

Rip-1 functions in association with one or more members of the steroid hormone receptor superfamily, and particularly, in association with one or more members of the glucocorticoid receptor (GR) family, and more particularly, in association with one or more members of the GR-type II receptor family. By “in association with” is meant that Rip-1 is a part of, forms a discrete complex with, or is functionally interactive or combined with, one or more of said steroid receptors. Thus, the Vpr, Rip-1, and steroid receptor or other component may be chemically and/or physically bound together to form a multi-part complex.

The cellular trafficking characteristics which have been observed for Rip-1 are consistent with Rip-1 functioning in association with, or even being a member of the steroid hormone receptor superfamily. The glucocorticoid and mineralocorticoid receptors are examples of members of this protein family which are known to translocate from the cytoplasm to the nucleus upon exposure to their ligand. Two types of glucocorticoid receptors have been described. Type I receptors are concentrated in the nucleus even when there is no ligand present. Type II receptors specifically concentrate in the cytoplasm in the absence of ligand, and only translocate to the nucleus in the presence of their appropriate stimulating hormone. The two types of glucocorticoid receptors have high affinity for their specific ligands, and are considered to function through the same transduction pathways. The main functional difference between these two classes of receptors is that the type II receptors are activated by their ligands in such a way that they only transactivate their target cellular protooncogenes in some, but not in all cells. Such cellular specificity is not observed in type I receptors. These observations are consistent with Rip-1 being functionally closely associated with, or actually being a GR-type II molecule.

Glucocorticoid receptors have a number of roles. Glucocorticoid receptors have been shown to act as powerful transactivators. Glucocorticoid receptors have also been shown to operate through the repression of gene expression for particular open reading frames. Glucocorticoid receptor mediated repression is attained by competition for the sites on the DNA molecule which would otherwise be bound by transactivators. An example of the latter is the specific bilateral relationship which has been described for glucocorticoid receptors and c-Jun. In this case, the glucocorticoid receptor represses c-Jun activity, and the opposite is also observed. The phorbol ester PMA has been reported to activate transcription of the AP-1/c-Jun promoter. In addition, glucocorticoids have been shown to counter lymphokine activity as observed by the inhibition of proliferation of a variety of cell lines. This mechanism is deemed to affect immunoregulatory mechanisms in areas such as T cell activation, which is in part mediated by the Jun/AP-1 activity, and its resulting lymphokines. The observation of a cessation in proliferation in different cell lines transfected with Vpr is considered explained by a glucocorticoid receptor mediated pathway, in which Rip-1, alone or in association with one or more steroid receptors or other components, or one or more steroid receptors, acts to bridge viral and cellular activities.

It is also important to note that the glucocorticoid receptors function as a part of a larger multimeric complex. These 330 KD protein clusters comprise a heat shock protein 90 dimer, a heat shock protein 56 unit, and sometimes by a heat shock protein 70 unit (HSP 70), in addition to the specific glucocorticoid receptor molecule; and Rip-1 has been observed in association with this HSP 70. The glucocorticoid receptor polypeptide itself is usually composed of three functional domains arranged in a linear configuration; a hormone binding domain, a DNA binding domain, and a third domain which has been shown to interact with additional cellular proteins, defining the trafficking characteristics of this gene product. It is contemplated that the complex comprising Rip-1, Vpr, and a steroid receptor or other components, may include as an example of the other components, the heat shock protein units described above.

Since Rip-1 in human cells appears to act in conjunction with a member of the steroid hormone receptor superfamily, especially the glucocorticoid receptor family, this may elucidate the manner in which the binding of Vpr to Rip-1 is involved in HIV replication and thus pathogenesis. Accordingly, interactively blocking Rip-1 or a complex including Rip-1 effectively inactivates Vpr and prevents it from converting cells to better HIV replication hosts. The identification of compounds which can inhibit the effects of Vpr and thereby inhibit HIV replication in HIV infected cells is based on the discovery that many of the actions of Vpr are analogous to those of a glucocorticoid. The mechanism of action of Vpr allows for the targeting of that mechanism for active intervention, and thereby the rational design and selection of anti-HIV compounds.

Rip-1 is the first Vpr associating protein which has been identified in accordance with the present invention, but it is possible that other gene products may either interact with Vpr directly, or indirectly through Rip-1 mediated associations. It has also been discovered in accordance with the present invention, that one or more steroid receptors, especially the glucocorticoid, and GR-type II receptors, may form a multi-part complex with, or are otherwise functionally interactive or combined with, Rip-1 and Vpr, whereby Vpr becomes translocated from the cytoplasm to the nucleus of the human host cell, and there plays an essential role in HIV replication.

U.S. Pat. No. 5,780,220, which is incorporated herein by reference, describes the treatment of individuals exposed to or infected with HIV, by administering to such individuals compounds which are steroid hormone receptor antagonists, particularly glucocorticoid receptor antagonists, and more particularly GR-type II receptor antagonists. Such receptor antagonists inhibit or prevent the replicative and other essential functions of Vpr by interactively blocking the Vpr target in human cells. The use of the glucocorticoid receptor antagonist mifeprestone, in the treatment of HIV infected individuals is set forth therein.

There remains a need to identify methods of treating individuals suffering from HIV infection. There remains a need to identify compounds which prevent or inhibit HIV replication in infected cells and thereby are useful for treating individuals suffering from HIV infection. There remains a need to identify methods of treating individuals who have been exposed to HIV to prevent them from becoming HIV infection. There remains a need to identify pharmaceutical compositions useful in such methods.

Eukaryotic cells and their viruses have evolved at least two mechanisms for recruiting and positioning ribosomes at the start sites for translation of RNA messages. The primary mechanism involves recognition of a 7-methyl guanosine cap on the 5′ terminus of the mRNA by a set of canonical initiation factors that recruit the 43S particle—including the 40S ribosomal subunit and eukaryotic initiation factor 3 (eIF3)—forming the 48S preinitiation complex (Merrick & Hershey, 1996; Pain, 1996; Sachs et al., 1997). Alternatively, numerous viruses and some eukaryotic mRNAs utilize a cap-independent pathway in which an RNA element, the internal ribosome entry site (IRES), drives preinitiation complex formation by positioning the ribosome on the message, either at or just upstream of the start site. In hepatitis C virus (HCV), the major infectious agent leading to non-A, non-B hepatitis, the minimum IRES includes nearly the entire 5′ untranslated region (UTR) of the message (for review, see Rijnbrand & Lemon, 2000). The secondary structure of the HCV IRES RNA, one of the most conserved regions of the entire viral genome, is critical for translation initiation, and is similar to that of the related pestiviruses and GB virus B (Brown et al., 1992; Wang et al., 1994, 1995; Le et al., 1995; Rijnbrand et al., 1995; Honda et al., 1996a, 1996b, 1999; Pickering et al., 1997; Varaklioti et al., 1998; Psaridi et al., 1999; Tang et al., 1999).

The 341-nucleotide 5′ non-translated region is the most conserved part of the hepatitis C virus (HCV) genome. It contains a highly structured internal ribosomal entry site (IRES) that mediates cap-independent initiation of translation of the viral polyprotein by a mechanism that is unprecedented in eukaryotes. The first step in translation initiation is assembly of eukaryotic initiation factor (elF) 3, eIF2, GTP, initiator tRNA and a 40S ribosomal subunit into a 43S preinitiation complex (Buratti et al., 1998, Kieft et al., 2001). The HCV IRES recruits this complex and directs its precise attachment at the initiation codon to form a 48S complex in a process that does not involve eIFs 4A, 4B or 4F. The IRES contains sites that bind independently with the eIF3 and 40S subunit components of 43S complexes, and structural determinants that ensure the correct spatial orientation of these binding sites so that the 48S complex assembles precisely at the initiation codon.

HCV IRES RNA adopts a specific three-dimensional fold in the presence of physiological concentrations of metal ions (Kieftet al., 1999). Rather than forming a tightly packed globular structure, the RNA helices extend from two folded helical junctions, suggesting that the IRES RNA acts as a structural scaffold in which specifically placed recognition sites recruit the translational machinery. This is supported by the observation that eIF3 and the 40S ribosomal subunit, the two largest components of the 43S particle, bind directly to the HCV IRES RNA (Pestova et al., 1998). Unlike IRESs found in some other RNA viruses, such as poliovirus, the IRES RNA•40S•eIF3 ternary pre-initiation complex forms without the involvement of other cellular factors (Pestova et al., 1998). Although several other proteins appear to interact with the HCVIRES RNA, they are not required for 43S binding to the IRES (Ali & Siddiqui, 1995, 1997; Yen et al., 1995; Hahm et al., 1998; Fukushi et al., 1999).

IRES/EIF/40S complexes have been reported to be important for other RNA viruses. Flavivruses [such as GBV-B, GBV-C, Japanese Encephalovirus (JEV) and West Nile Virus (WNV)] (Malancha & Sudhanshu, 2000, Blackwell & Brinton, 2000) as well as pestiviruses [such as classical swine fever virus (CSFV), border disease virus (BDV), and bovine viral disease virus (BVDV)] (Sizova et al, 1998, Pestova et al., 1998, Fletcher et al., 2002) and picornoviruses [such as poliovirus, Foot and mouth disease virus (FMDV) and encephalomyocarditis virus (EMCV)] (Jang et al., 1988, Pelletier & Sonenberg, 1988) and calicivirus [such as the Norwalk virus] (Daughenbaugh et al. 2003). Similar ribosomal binding sites on coronaviruses have been reported to be important for RNA translation, replication, or transcription (O'Connor & Brian, 2000, Raman et al, 2003).

Furthermore, HSV gene expression is characterized by a temporal pattern of expression of three gene classes: immediate early (IE), early (E), and late (L) genes. IE genes are transcribed in the absence of de novo viral protein synthesis, E genes are activated by IE gene products, and L genes are activated by viral DNA synthesis (reviewed in Roizman and Knipe, 2001). The IE-infected cell protein 27 (ICP27) is essential for viral replication and expression of certain early and nearly all late viral genes (Rice et al., 1989, Sacks et al., 1985 and Uprichard and Knipe, 1996). ICP27 is a multi-functional protein in that it increases late viral gene transcription (Jean et al., 2001), binds to RNA (Mears and Rice, 1996), associates with RNA pol II (Zhou and Knipe, 2002), and shuttles from the nucleus to the cytoplasm (Mears and Rice, 1998 and Soliman et al., 1997). ICP27 has been shown to associate with cellular transcriptional proteins (Taylor and Knipe, 2004 and Zhou and Knipe, 2002), as well as viral transcriptional proteins ICP4 (Panagiotidis et al., 1997) and ICP8 (Taylor and Knipe, 2004 and Zhou and Knipe, 2002), and function in post-transcriptional processes, such as pre-mRNA splicing and mRNA export, through its interactions with cellular splicing and export factors involved in these pathways (Koffa et al., 2001). ICP27 directly affects the expression and stability of specific viral and cellular transcripts in both transfected (Brown et al., 1995) and infected cells (Cheung et al., 2000, Ellison et al., 2000 and Pearson et al., 2004). Furthermore, ICP27 is thought to function, along with the virion host shut-off (vhs) protein, in shut-off of cellular protein synthesis (Sacks et al., 1985 and Song et al., 2001), and the involvement of ICP27 in inhibition of pre-mRNA splicing provides a mechanism for shut-off of cellular protein synthesis (Sandri-Goldin, 1998). Proteomic studies involving immunoprecipitation of ICP27 and mass spectrometric identification of co-precipitated proteins show an association of ICP27 with the cellular translation initiation factors poly A binding protein (PABP), eukaryotic initiation factor 3 (eIF3), and eukaryotic initiation factor 4G (eIF4G) in infected cells (Fontaine-Rodriguez et al, 2004). Immunoprecipitation-western blot studies confirmed these associations. Finally, purified MBP-tagged ICP27 (MBP-27) can interact with eIF3 subunits p47 and p116 in vitro. These results show that ICP27 may play a role in stimulating translation of certain viral and host mRNAs and/or in inhibiting host mRNA translation.

The interaction of eIF4G and PABP is thought to facilitate the interaction between the 5′ cap and 3′ polyadenylated end of the mRNA, which enhances translation both in vitro and in vivo, and facilitates recruitment of the 40S ribosomal subunit to the 5′ end of the mRNA molecule [(reviewed in Prevot et al., 2003) and (Sonenberg and Dever, 2003)]. eIF3 is a multi-subunit component of the 40S ribosome, and interaction of eIF4G with eIF3 leads to recruitment of mRNA to the 43S complex (reviewed (Gallie, 2002). Thus, the interaction of ICP27 with both eIF3 and PABP could lead to the recruitment of these translation initiation factors to viral mRNA and stimulation of translation of these mRNAs. Moreover, both PABP and eIF3 p47 subunit have been shown to localize to both the cytoplasm and the nucleus (Afonina et al., 1998 and Shi et al., 2003). Therefore, ICP27 could recruit these proteins to nascent viral transcripts, which may facilitate viral mRNA export out of the nucleus, and increase the efficiency of translational initiation on these mRNAs.

PABP, eIF3, and eIF4G are known targets for modification by viruses. These cellular translation factors are altered by specific viral proteins, and as a result, host cell protein synthesis is shut down (reviewed in Bushell and Sarnow, 2002 and Daughenbaugh et al., 2003). Translation initiation factor eIF4G acts as a scaffolding protein for the cap-binding complex (eIF4F), and interacts with multiple translation initiation proteins including PABP and eIF3 (reviewed in Kawaguchi and Bailey-Serres, 2002). Furthermore, each of these translation initiation factors have been shown to function in viral translation regulatory mechanisms, which require specific binding to viral proteins (reviewed in Gallie, 2002).

Using a yeast two-hybrid system, the cDNA of a Vpr-interacting cellular factor, termed human Vpr Interacting Protein (hVIP/mov34) was cloned (Mahalingam et al., 1998) hVIP/mov34 has complete homology with a reported member of the eIF3 complex (Asano et al., 1997). eIF3 is a large multimeric complex that regulates transcriptional events and is essential for G1/S and G2/M phase progression through the cell cycle. hVIP is thought to be a GR-responsive protein. Experimental results strongly suggest that hVIP is associated with the activated glucocorticoid receptor complex.

Glucocorticoids regulate diverse functions and are important to maintain central nervous system, cardiovascular, metabolic, and immune homeostasis. They also exert anti-inflammatory and immunosuppressive effects, which have made them invaluable therapeutic agents in numerous diseases (Chrousos, 1995). The actions of these hormones are mediated by their specific intracellular receptors, such as the GR. Several host co-activators of the GR have been described that directly interact with GR and components of the transcription initiation complex to enhance the glucocorticoid signal to the transcription machinery (Shibata et al., 1997).

The GR is the prototypic member of the translocating class of steroid receptors that are ubiquitously expressed in almost all human tissues and organs. Unliganded GR is found in the cytoplasm and moves rapidly into the nucleus in response to hormone stimulation (Htun et al., 1996, McNally et al., 2000). GR interacts in the cytoplasm with a complex array of chaperone proteins, including HSP90 and HSP70, and ligand-dependent displacement of these proteins is thought to be intimately involved in the translocation process (Bamberger et al., 1996, Beato et al., 1996). Both GR and hVIP are known Vpr ligands. Steroid hormone receptor antagonists such as mifepristone prevent the GR from moving into the nucleus in response to appropriate stimulation. In addition, mifepristone blocks the Vpr-induced nuclear entry of hVIP. HVIP had been reported as a potential Vpr ligand and demonstrated its role in cell cycle regulation as antisense of this gene induced cell cycle arrest at the G2/M phase (Mahalingam et al., 1998).

Glucocorticoids have been demonstrated to mimic the effects of Vpr and glucocorticoid antagonists mifepristone has been shown to revert these effects of Vpr (Ayyavoo et al., 1997, Ayyavoo et al., 2002, Kino et al., 1999, Sherman et al., 2000). Moreover, mifepristone has been shown to block the nuclear translocation of hVIP induced by Vpr in cells. This result clearly demonstrates that mifepristone inhibits the translocation of hVIP induced by the expression of Vpr and strongly suggested that mifepristone and other GR antagonists can directly effect hVIP/mov34. In addition, these results implicate the use of other drug compounds to block/inhibit EIF3/mov34 (antisense, antibodies, inhibitory RNA) as a potential treatment for viral pathogens like Hepatitis C virus.

### SUMMARY OF THE INVENTION

The present invention further relates to pharmaceutical composition comprising: Polyethylene Glycol (PEG) and a compound that inhibits viral replication in humans, the compound having a structure selected from the group consisting of Formulas D1-D21, and pharmaceutically acceptable salts thereof.

The present invention further relates to methods of treating an individual who has been infected with viral infections, including HIV, HCV, and HSV. The method comprise the step of administering to the individual an amount of a pharmaceutical composition comprising a PEG and a compound that inhibits viral replication having a structure selected from the group consisting of Formulas D1-D21, and pharmaceutically acceptable salts thereof effective to inhibit viral replication in the individuals.

The present invention further relates to methods of preventing viral infection in an individual at an elevated risk of becoming infected. The method comprises the step of administering to the individual a prophylactically effective amount of a pharmaceutical composition that comprises PEG or diluent, and, a compound that inhibits viral replication having a structure selected from the group consisting of Formulas D1-D21, and pharmaceutically acceptable salts thereof effective to inhibit viral replication.

### DETAILED DESCRIPTION OF PREFERRED EMBODIMENTS

The present invention provides delivering pharmaceutical compositions comprising a compound having a structure selected from the group consisting of Formulas D1-D21, and pharmaceutically acceptable salts thereof using a PEG as a carrier. The present invention provides methods of treating individuals infected with HIV, HCV, and HSV by administering to them a therapeutically effective amount of such compositions. The present invention further provides methods of preventing viral infection in individuals exposed to these viruses, by administering to them a prophylactically effective amount of such compositions.

The compounds of the invention may act as steroid hormone receptor antagonists that interactively blocks Rip-1, alone or in association with one or more steroid receptors, or other components, or one or more steroid receptors alone, preventing or inhibiting formation and translocation of the Rip-1 and/or steroid receptor or other component complex.

As used herein, the term “high risk individual” is meant to refer to an individual who is suspected of having been exposed to the viruses. Such individuals include health care or other individuals who may have accidently exchanged blood with an infected individual, such as through an accidental needle stick, injuries that occur during emergency medical care, rescue or arrest and unprotected sexual contact. High risk individuals can be treated prophylactically before any detection of viral infection can be made.

As used herein, the term “therapeutically effective amount” is meant to refer to an amount of a compound which produces a medicinal effect observed as reduction or reverse in viral titer and/or and increase or stabilization of immune cells when a therapeutically effective amount of a compound is administered to an individual who is infected with the viruses. Therapeutically effective amounts are typically determined by the effect they have compared to the effect observed when a composition which includes no active ingredient is administered to a similarly situated individual.

As used herein, the term “viral replication” is meant to refer to a replication of disease causing viruses in human body. These viruses include, but are not limited to, HIV, HCV, and HSV.

As used herein, the term a “prophylactically effective amount” is meant to refer to an amount of a compound which produces a medicinal effect observed as the prevention of an infection in an individual when a prophylactically effective amount of a compound is administered to a high risk individual. Prophylactically effective amounts are typically determined by the effect they have compared to the effect observed when a composition which includes no active ingredient is administered to a similarly situated individual.

The invention provides novel pharmaceutical compositions comprising of novel ways to formulate antiviral compounds that are inhibitors of viral replication. The antiviral compounds included in the pharmaceutical compositions of the present invention have a formula selected from the group consisting of Formulas D1-D21, as set forth below, or a pharmaceutically acceptable salt thereof. The invention provides novel pharmaceutical compositions comprising antiviral compositions that inhibit viral replication. In some preferred embodiments, the VIRAL replication inhibitor in the pharmaceutical compositions of the present invention has a formula of Formula 1 as set forth in the section below entitled Formulae. In some preferred embodiments, the VIRAL replication inhibitor in the pharmaceutical compositions of the present invention has a formula of Formula 2 as set forth in the section below entitled Formulae. In some preferred embodiments, the VIRAL replication inhibitor in the pharmaceutical compositions of the present invention has a formula of Formula 3 as set forth in the section below entitled Formulae. In some preferred embodiments, the VIRAL replication inhibitor in the pharmaceutical compositions of the present invention has a formula of Formula 4 as set forth in the section below entitled Formulae. In some preferred embodiments, the VIRAL replication inhibitor in the pharmaceutical compositions of the present invention has a formula of Formula 5 as set forth in the section below entitled Formulae. In some preferred embodiments, the VIRAL replication inhibitor in the pharmaceutical compositions of the present invention has a formula of Formula 6 as set forth in the section below entitled Formulae. In some preferred embodiments, the VIRAL replication inhibitor in the pharmaceutical compositions of the present invention has a formula of Formula 7 as set forth in the section below entitled Formulae. In some preferred embodiments, the VIRAL replication inhibitor in the pharmaceutical compositions of the present invention has a formula of Formula 8 as set forth in the section below entitled Formulae. In some preferred embodiments, the VIRAL replication inhibitor in the pharmaceutical compositions of the present invention has a formula of Formula 9 as set forth in the section below entitled Formulae. In some preferred embodiments, the VIRAL replication inhibitor in the pharmaceutical compositions of the present invention has a formula of Formula 10 as set forth in the section below entitled Formulae. In some preferred embodiments, the VIRAL replication inhibitor in the pharmaceutical compositions of the present invention has a formula of Formula 11 as set forth in the section below entitled Formulae. In some preferred embodiments, the VIRAL replication inhibitor in the pharmaceutical compositions of the present invention has a formula of Formula 12 as set forth in the section below entitled Formulae. In some preferred embodiments, the VIRAL replication inhibitor in the pharmaceutical compositions of the present invention has a formula of Formula 13 as set forth in the section below entitled Formulae. In some preferred embodiments, the VIRAL replication inhibitor in the pharmaceutical compositions of the present invention has a formula of Formula 14 as set forth in the section below entitled Formulae. In some preferred embodiments, the VIRAL replication inhibitor in the pharmaceutical compositions of the present invention has a formula of Formula 15 as set forth in the section below entitled Formulae. In some preferred embodiments, the VIRAL replication inhibitor in the pharmaceutical compositions of the present invention has a formula of Formula 16 as set forth in the section below entitled Formulae. In some preferred embodiments, the VIRAL replication inhibitor in the pharmaceutical compositions of the present invention has a formula of Formula 17 as set forth in the section below entitled Formulae. In some preferred embodiments, the VIRAL replication inhibitor in the pharmaceutical compositions of the present invention has a formula of Formula 18 as set forth in the section below entitled Formulae. In some preferred embodiments, the VIRAL replication inhibitor in the pharmaceutical compositions of the present invention has a formula of Formula 19 as set forth in the section below entitled Formulae. In some preferred embodiments, the VIRAL replication inhibitor in the pharmaceutical compositions of the present invention has a formula of Formula 20 as set forth in the section below entitled Formulae. In some preferred embodiments, the VIRAL replication inhibitor in the pharmaceutical compositions of the present invention has a formula of Formula 21 as set forth in the section below entitled Formulae.

In some embodiments the method of the invention additionally includes the use of the viral replication inhibitor compositions of the invention in combination with other methodologies to treat viral infections. In some embodiments, the viral replication inhibitor is administered in conjunction with other antiviral agents such as zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, lamivudine, ribavirin, peginterferon interferon alpha-2a, and interferon alpha-2b, alfa-2a, and peginterferon alfa-2b.

The pharmaceutical compositions comprising viral replication inhibitor compositions of the present invention may be administered by any means that enables the active agent to reach the agent's site of action in the body of the individual. Pharmaceutical compositions of the present invention may be administered by conventional routes of pharmaceutical administration. Pharmaceutical compositions may be administered parenterally, i.e. intravenous, subcutaneous, intramuscular. In some embodiments, the pharmaceutical compositions are administered orally. In some embodiments, the pharmaceutical compositions are administered transdermally and subdermally. Pharmaceutical compositions are administered to the individual for a length of time effective to eliminate, reduce or stabilize viral titer and/or increase or stabilize immune cell counts. When used prophylactically, Pharmaceutical compositions are administered to the individual for a length of time during which monitoring for evidence of infection continues.

Pharmaceutical compositions of the present invention may be administered either as individual therapeutic agents or in combination with other therapeutic agents. They can be administered alone, but are generally administered with a pharmaceutical carrier selected on the basis of the chosen route of administration and standard pharmaceutical practice.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.001 to 1 grams per kilogram of body weight, in some embodiments about 0.1 to 100 milligrams per kilogram of body weight. Ordinarily dosages are in the range of 0.5 to 50 milligrams per kilogram of body weight, and preferably 1 to 10 milligrams per kilogram per day. In some embodiments, the pharmaceutical compositions are given in divided doses 1 to 6 times a day or in sustained release form is effective to obtain desired results.

Dosage forms (composition) suitable for internal administration generally contain from about 1 milligram to about 500 milligrams of active ingredient per unit. In these pharmaceutical compositions the active ingredient will ordinarily be present in an amount of about 0.5-95 by weight based on the total weight of the composition. Generally, multiple administrations are performed.

Pharmaceutical compositions may be formulated by one having ordinary skill in the art with compositions selected depending upon the chosen mode of administration. Suitable pharmaceutical carriers are described in Remington's Pharmaceutical Sciences, A. Osol, a standard reference text in this field, which is incorporated herein by reference.

For parenteral and injectible administration, the compound can be formulated as a solution, suspension, emulsion or lyophilized powder in an association with PEG, including PEG-400 along with other pharmaceutically acceptable carriers. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. In some embodiments, a parenteral composition suitable for administration by injection is prepared by dissolving 1.5% by weight of active ingredient in 0.9% sodium chloride solution.

According to some embodiments of the present invention, the composition is administered to tissue of an individual by topically or by lavage. The compounds may be formulated as a cream, ointment, salve, douche, suppository or solution for topical administration or irrigation in an association with PEG, including PEG-400 along with other pharmaceutically acceptable carriers. Formulations for such routes administration of pharmaceutical compositions are well known. Generally, additives for isotonicity can include sodium chloride, dextrose, mannitol, sorbitol and lactose.

In some cases, isotonic solutions such as phosphate buffered saline are used. Stabilizers include gelatin and albumin. In some embodiments, a vasoconstriction agent is added to the formulation. The pharmaceutical preparations according to the present invention are preferably provided sterile and pyrogen free. The pharmaceutical preparations according to the present invention which are to be used as injectables are provided sterile, pyrogen free and particulate free.

A pharmaceutically acceptable formulation will provide the active ingredient(s) in proper physical form together with such excipients, diluents, stabilizers, preservatives and other ingredients as are appropriate to the nature and composition of the dosage form and the properties of the drug ingredient(s) in the formulation environment and drug delivery system.

In some embodiments, the invention relates to methods of treating patients suffering from HIV infection. In some embodiments, the invention relates to methods of preventing HIV infection in high risk individuals. In some embodiments, the invention relates to methods of treating patients suffering from HCV infection. In some embodiments, the invention relates to methods of preventing HCV infection in high risk individuals. In some embodiments, the invention relates to methods of treating patients suffering from HSV infection. In some embodiments, the invention relates to methods of preventing HSV infection in high risk individuals.

According to some embodiments of the invention, the patient is treated with other antiviral therapy in conjunction the administration of pharmaceutical compositions according to the invention. The use of multiple therapeutic approaches provides the patient with a broader based intervention.

According to some aspects of the present invention, in combination with administration of the composition that comprises the HIV replication inhibitor, the individual is also administered another agent. In some embodiments, in combination with administration of the composition, the individual additionally receives compositions that comprises mifepristone, zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, lamivudine, ribavirin, peginterferon interferon alpha-2a, and interferon alpha-2b, alfa-2a, and peginterferon alfa-2b.

Other antivirals may also be used delivered according to standard protocols using standard agents, dosages and regimens. In some embodiments, the pharmaceutical compositions contain one or more of the compounds selected from the group consisting of Formulas D1-D21, and pharmaceutically acceptable salts thereof. In some embodiments, the pharmaceutical compositions contain one or more of the compounds selected from the group consisting of Formula D1-D21, and pharmaceutically acceptable salts thereof and at least one additional antiviral selected from the group consisting of: mifepristone, zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, lamivudine, ribavirin, peginterferon interferon alpha-2a, and interferon alpha-2b, alfa-2a, and peginterferon alfa-2b, together with a pharmaceutically acceptable carrier.

The pharmaceutical compositions according to the present invention may be administered as a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously.

Additionally, the present invention is particularly useful to prevent recurrence of infection in patients who have been previously diagnosed as HIV positive but show no indication of infection.

Those having ordinary skill in the art can readily identify high risk individuals. Healthcare workers come into contact with infected blood and suffer needle sticks from syringes used on virally infected individuals. Surgeons cut themselves during surgery. Lab workers, dentists and dental technicians come into contact with infected blood as do emergency medical and rescue workers and law enforcement officers. Individuals involved in athletics and sexually active individuals can also become exposed to the virus. Once any person comes into contact with infected blood, that individual is at an elevated risk of infection.

The present invention is not limited to any particular theory or mechanism of action and while it is currently believed that the compounds identified herein operate through blocking the steroid hormone receptor complex that comprises Rip-1, such explanation of the mechanism of action is not intended to limit the invention. The present invention is further illustrated by the following examples, which are not intended to be limiting in any way.

### REFERENCES

Ackermann et al., 1984 M. Ackermann, D. K. Braun, L. Pereira and B. Roizman, Characterization of herpes simplex virus 1 alpha proteins 0, 4, and 27 with monoclonal antibodies, J. Virol. 52 (1984), pp. 108-118.
Afonina et al., 1998 E. Afonina, R. Stauber and G. N. Pavlakis, The human poly(A)-binding protein 1 shuttles between the nucleus and the cytoplasm, J. Biol. Chem. 273 (1998), pp. 13015-13021
Ali N, Siddiqui A. 1995. Interaction of polypyrimidine tract-binding protein with the 59 noncoding region of the hepatitis C virus RNA genome and its functional requirement in internal initiation of translation. J Virol 69:6367-6375.
Ali N, Siddiqui A. 1997. The La antigen binds 59 noncoding region of the hepatitis C virus RNA in the context of the initiator AUG codon and stimulates internal ribosome entry site-mediated translation. Proc Natl Acad Sci USA 94:2249-2254.
Asano, K., Vomlocher, H. P., Richter-Cook, N. J., Merricj, W. C., Hinnebusch, A. G., and Hershey, W. B. (1997) J. Biol. Chem. 272, 27042-27052.
Asano et al., 1997 K. Asano, H. P. Vomlocher, N. J. Richter-Cook, W. C. Merrick, A. G. Hinnebusch and J. W. Hershey, Structure of cDNAs encoding human eukaryotic initiation factor 3 subunits. Possible roles in RNA binding and macromolecular assembly, J. Biol. Chem. 272 (1997), pp. 27042-27052.
Ayyavoo, V., Mahboubi, A., Mahalingam, S., Ramalingam, R., Kudchodkar, S., Williams, W. V., Green, D. R., and Weiner, D. B. (1997) Nat. Med. 3, 1117-1123.
Ayyavoo, V., Muthumani, K., Kudchodkar, S. B., Zhang, D., Ramanathan, M. P., Dayes, N. S., Kim, J. J., Sin, J. I., Montaner, L. J., and Weiner, D. B. (2002) Int. Immunol. 14, 13-22.
Bamberger, C. M., Schulte, H. M., and Chrousos, G. P. (1996) Endocr. Rev. 17, 245-261.
Beato, M., and Sanchez-Pacheco, A. (1996) Endocr. Rev. 17, 587-609.
Blackwell, J L and Brinton, M A. 1997. Translation elongation factor-1alpha interacts with the 3′ stem-loop region of West Nile Virus genomic RNA. J Virol 71:6433-6444.
Brown et al., 1995 C. R. Brown, M. S. Nakamura, J. D. Mosca, G. S. Hayward, S. E. Straus and L. P. Perera, Herpes simplex virus trans-regulatory protein ICP27 stabilizes and binds to 3′ ends of labile mRNA, J. Virol. 69 (1995), pp. 7187-7195.
Brown E S, Zhang H, Ping L H, Lemon S M. 1992. Secondary structure of the 59 nontranslated regions of hepatitis C virus and pestivirus genomic RNAs. Nucleic Acids Res 20:5041-5045.
Buratti E, Tisminetzky S, Zotti M, Baralle F E. 1998. Functional analysis of the interaction between HSV 59 UTR and putative subunits of eukaryotic initiation factor eIF3. Nucleic Acids Res 26:3179-3187.
Bushell and Sarnow, 2002 M. Bushell and P. Sarnow, Hijacking the translation apparatus by RNA viruses, J. Cell Biol. 158 (2002), pp. 395-399.
Chantal Petit, A. J. et al (1987) J. Clin. Invest. 79:1883
Chen et al., 2002 I. H. Chen, K. S. Sciabica and R. M. Sandri-Goldin, ICP27 interacts with the RNA export factor Aly/REF to direct herpes simplex virus type 1 intronless mRNAs to the TAP export pathway, J. Virol. 76 (2002), pp. 12877-12889.
Cheung et al., 2000 P. Cheung, K. S. Ellison, R. Verity and J. R. Smiley, Herpes simplex virus ICP27 induces cytoplasmic accumulation of unspliced polyadenylated alpha-globin pre-mRNA in infected HeLa cells, J. Virol. 74 (2000), pp. 2913-2919.
Chrousos, G. P. (1995) N. Engl. J. Med. 332, 1351-1362.
Cohen, E. A., et al. (1990a) J. Virol. 64:3097-3099
Croxatto, H B, Salvatierra A M, Croxatto, H D, Fuentealba B. Effects of continuous treatment with low dose mifepristone throughout one menstrual cycle. Human Reprod. 1992. 7:945-50.
Daughenbaugh et al., 2003 K. F. Daughenbaugh, C. S. Fraser, J. W. Hershey and M. E. Hardy, The genome-linked protein VPg of the Norwalk virus binds eIF3, suggesting its role in translation initiation complex recruitment, EMBO J. 22 (2003), pp. 2852-2859.
Daughenbaugh, K F. et al. 2003. EMBO J. The genome-linked protein VPg of the Norwalk virus binds eIF3, suggesting its role in translation initiation complex recruitment. 22:2852-2859.
Dedera, D., et al. (1989) Virol. 63:3205-3208
Ellison et al., 2000 K. S. Ellison, S. A. Rice, R. Verity and J. R. Smiley, Processing of alpha-globin and ICP0 mRNA in cells infected with herpes simplex virus type 1 ICP27 mutants, J. Virol. 74 (2000), pp. 7307-7319.
Fletcher, S P et al. 2002. Pestivirus internal ribosome entry site (IRES) structure and function: Elements in the 5′ untranslated region important for IRES function. J Virol 76: 5024-5033.
Foldesi, I., G. Falkay and L. Kovacs, Determination of RU486 (mifepristone) in blood by radioreceptor assay: a pharmacokinetic study. Contraception 56 (1996), pp. 27-32.
Fontaine-Rodriguez et al, 2004 T. J. Taylor, M. Olesky and D. M. Knipe, Proteomics of herpes simplex virus infected cell protein 27: association with translation initiation factors, J. Virol. 330 (2004), pp. 487-492. Abstract-
Fukushi S, Okada M, Kageyama T, Hoshino F B, Katayama K. 1999. Specific interaction of a 25-kilodalton cellular protein, a 40S ribosomal subunit protein, with the internal ribosome entry site of hepatitis C virus genome. Virus Genes 19:153-161.
Gallie, 2002 D. R. Gallie, Protein-protein interactions required during translation, Plant Mol. Biol. 50 (2002), pp. 949-970.
Gallo, R. C. et al. (1984) Science 224:500
Griffin, G. E. et al. (1988) Nature 339:70
Hahm B, Kim Y K, Kim T Y, Jang S K et al., 1998. Heterogeneous nuclear ribonucleoprotein L interacts with the 3′ border of the internal ribosomal entry site of hepatitis C virus. J. Virol. 1998; 72(11):8782-8.
Hardwicke and Sandri-Goldin, 1994 M. A. Hardwicke and R. M. Sandri-Goldin, The herpes simplex virus regulatory protein ICP27 contributes to the decrease in cellular mRNA levels during infection, J. Virol. 68 (1994), pp. 4797-4810.
Hattori, N., et al. (1990) Proc. Natl. Acad. Sci. USA 87:8080-8084
Heikinheimo, O., Kekkone R. Dose-response relationships of RU486. Ann Med. 1993. 25: 71-6.
Heikinheimo, O., Kontula K, H. Croxatto Spitz I, Luukkainen T, Lahteenmaki P Pharmacokinetics of the antiprogestin RU486 in women during multiple doses administration. J. Steroid Biochem 32 (1989), pp. 22-25.
Honda M, Beard M R, Ping L H, Lemon S M. 1999. A phylogenetically conserved stem-loop structure at the 59 border of the internal ribosome entry site of hepatitis C virus is required for cap-independent viral translation. J Virol 73:1165-1174.
Honda M, Brown E A, Lemon S M. 1996a. Stability of a stem-loop involving the initiator AUG controls the efficiency of internal initiation of translation on hepatitis C virus RNA. RNA 2:955-968.
Honda M, Ping L H, Rijnbrand R C, Amphlett E, Clarke B, Rowlands D, Lemon S M. 1996b. Structural requirements for initiation of translation by internal ribosome entry within genome-length hepatitis C virus RNA. Virology 222:31-42.
Htun, H., Barsony, J., Renyi, I., Gould, D. L., and Hager, G. L. (1996) Proc. Natl. Acad. Sci. U.S.A. 93, 4845-4850.
Jang et al. 1988. A segment of the 5′ nontranslated region of encephalomyocarditis virus RNA directs internal entry of ribosomes during in vitro translation, J. Virol. 62: 2636-2643.
Jean et al., 2001 S. Jean, K. M. LeVan, B. Song, M. Levine and D. M. Kipe, Herpes simplex virus 1 ICP27 is required for transcription of two viral late (gamma2) genes in infected cells, Virology 283 (2001), pp. 273-284.
Kawaguchi and Bailey-Serres, 2002 R. Kawaguchi and J. Bailey-Serres, Regulation of translational initiation in plants, Curr. Opin. Plant Biol. 5 (2002), pp. 460-465.
Kekkonen, R., O. Heikinheimo, E. Mandelin and P. Lahteenmaki, Pharmacokinetics of mifepristone after low oral doses. Contraception 54 (1996), pp. 229-234.
Kieft J S, Zhou K, Jubin R, Doudna J A. 2001. Mechanism of ribosome recruitment by hepatitis C virus IRES. RNA. 7:194-206.
Kieft J S, Zhou K, Jubin R, Murray M G, Lau J Y, Doudna J A. 1999. The hepatitis C virus internal ribosome entry site adopts an ion-dependent tertiary fold. J Mol Biol 292:513-529.
Kino, T., Gragerov, A., Kopp, J. B., Stauber, R. H., Pavlakis, G. N., and Chrousos, G. P. (1999) J. Exp. Med. 189, 51-62.
Koffa et al., 2001 M. D. Koffa, J. B. Clements, E. Izaurralde, S. Wadd, S. A. Wilson, I. W. Mattaj and S. Kuersten, Herpes simplex virus ICP27 protein provides viral mRNAs with access to the cellular mRNA export pathway, EMBO J. 20 (2001), pp. 5769-5778.
Le S Y, Sonenberg N, Maizel J V Jr. 1995. Unusual folded regions and ribosome landing pad within hepatitis C virus and pestivirus RNAs. Gene 154:137-143.
Levy, D. N. et al. (1993) Cell 72:541
Levy, J. A. et al. (1984) Science 225:840
Li, G. et al. (1993) J. Virol. 67:3969
Mahalingam, S., Ayyavoo, V., Patel, M., Kieber-Emmons, T., Kao, G. D., Muschel, R. J., and Weiner, D. B. (1998) Proc. Natl. Acad. Sci. U.S.A. 98, 3419-3424.
Malancha, T A, and Sudhanshu, V., 2000. Mov34 protein from mouse brain interacts with the 3′ noncoding region of Japanese encephalitis virus. J Virol 74: 5108-5115.
McLauchlan et al., 1992 J. McLauchlan, A. Phelan, C. Loney, R. M. Sandri-Goldin and J. B. Clements, Herpes simplex virus IE63 acts at the posttranscriptional level to stimulate viral mRNA 3′ processing, J. Virol. 66 (1992), pp. 6939-6945.
McNally, J. G., Muller, W. G., Walker, D., Wolford, R., and Hager, G. L. (2000) Science 287, 1262-1265.
Mears and Rice, 1996 W. E. Mears and S. A. Rice, The RGG box motif of the herpes simplex virus ICP27 protein mediates an RNA-binding activity and determines in vivo methylation, J. Virol. 70 (1996), pp. 7445-7453.
Mears and Rice, 1998 W. E. Mears and S. A. Rice, The herpes simplex virus immediate-early protein ICP27 shuttles between nucleus and cytoplasm, Virology 242 (1998), pp. 128-137.
Merrick W C & Hershey J W, 1996. Conservation and diversity in the structure of translation initiation factor EIF3 from humans and yeast. (1996) Biochimie. 1996; 78 (11-12): 903-7.
Methot et al., 1997 N. Methot, E. Rom, H. Olsen and N. Sonenberg, The human homologue of the yeast Prt1 protein is an integral part of the eukaryotic initiation factor 3 complex and interacts with p170, J. Biol. Chem. 272 (1997), pp. 1110-1116.
Myers, G. et al. (1992) AIDS Res. Hum. Retrovir. 8:373
O'Connor J B, Brian D A 2000. Virology. 269:172-82.
Ogawa, K., et al. (1989) J. Virol. 63:4110-4114
Pain V M, Morley S J 1996. Translational regulation during activation of porcine peripheral blood lymphocytes: association and phosphorylation of the alpha and gamma subunits of the initiation factor complex eIF-4F. Biochem J. 1995 Dec. 1; 312 (Pt 2):627-35.
Panagiotidis et al., 1997 C. A. Panagiotidis, E. K. Lium and S. J. Silverstein, Physical and functional interactions between herpes simplex virus immediate-early proteins ICP4 and ICP27, J. Virol. 71 (1997), pp. 1547-1557.
Pearson et al., 2004 A. Pearson, D. M. Knipe and D. M. Coen, ICP27 selectively regulates the cytoplasmic localization of a subset of viral transcripts in herpes simplex virus type 1-infected cells, J. Virol. 78 (2004), pp. 23-32.
Pelletier, J., Sonenberg, N. 1988. Internal initiation of translation of eukaryotic mRNA directed by a sequence derived from poliovirus RNA. Nature. 334: 320-325.
Perkins et al., 2003 K. D. Perkins, J. Gregonis, S. Borge and S. A. Rice, Transactivation of a viral target gene by herpes simplex virus ICP27 is posttranscriptional and does not require the endogenous promoter or polyadenylation site, J. Virol. 77 (2003), pp. 9872-9884.
Pestova T V, Shatsky I N, Fletcher S P, Jackson R J, Hellen C U T. 1998. Aprokaryotic-like mode of cytoplasmic eukaryotic ribosome binding to the initiation codon during internal translation initiation of hepatitis C and classical swine fever virus RNAs. Genes & Dev 12:67-83.
Pickering J M, Thomas H C, Karayiannis P. 1997. Predicted secondary structure of the hepatitis G virus and GB virus-A 59 untranslated regions consistent with an internal ribosome entry site. J Viral Hepat 4:175-184.
Prevot et al., 2003 D. Prevot, J. L. Darlix and T. Ohlmann, Conducting the initiation of protein synthesis: the role of eIF4G, Biol. Cell 95 (2003), pp. 141-156.
Psaridi L, Georgopoulou U, Varaklioti A, Mavromara P. 1999. Mutationalanalysis of a conserved tetraloop in the 59 untranslated region of hepatitis C virus identifies a novel RNA element essential for the internal ribosome entry site function. FEBS Lett 453:49-53.
Raman S, Bouma P, Williams G D, Brian D A. 2003. J. Virol. 77:6720-30.
Rice and Knipe, 1990 S. A. Rice and D. M. Knipe, Genetic evidence for two distinct transactivation functions of the herpes simplex virus alpha protein ICP27, J. Virol. 64 (1990), pp. 1704-1715.
Rice et al., 1989 S. A. Rice, L. S. Su and D. M. Knipe, Herpes simplex virus alpha protein ICP27 possesses separable positive and negative regulatory activities, J. Virol. 63 (1989), pp. 3399-3407.
Rich, E. A. et al. (1992) J. Clin. Invest. 89:176
Rijnbrand R, Bredenbeek P, van der Straaten T, Whetter L, Inchauspe G, Lemon S, Spaan W. 1995. Almost the entire 59 non-translated region of hepatitis C virus is required for cap-independent trans-lation. FEBS Lett 365:115-119.
Rijnbrand R C, Lemon S M. 2000. Internal ribosome entry site-mediated translation in hepatitis C virus replication. Curr Top Microbiol Immunol 242:85-116.
Roizman and Knipe, 2001 B. Roizman and D. M. Knipe, Herpes simplex viruses and their replication In: D. M. Knipe and P. M. Howley, Editors, Fields Virology (4th ed.), Lippincott, Williams and Wilkins, Philadelphia, Pa. (2001), pp. 2399-2460.
Rose, R. M. et al. (1986) Am. Rev. Respir. Dis. 143:850
Roulston, A. et al. (1992) J. Exp. Med. 175:751
Sachs G, Bayle D, Weeks D et al., 1997. Identification of membrane insertion sequences of the rabbit gastric cholecystokinin-A receptor by in vitro translation. (1997) J Biol Chem 1997; 272(32):19697-707.
Sacks et al., 1985 W. R. Sacks, C. C. Greene, D. P. Aschman and P. A. Schaffer, Herpes simplex virus type 1 ICP27 is an essential regulatory protein, J. Virol. 55 (1985), pp. 796-805.
Salahuddin, S. Z. et al. (1986) Blood 68:281
Sandri-Goldin, 1998 R. M. Sandri-Goldin, ICP27 mediates HSV RNA export by shuttling through a leucine-rich nuclear export signal and binding viral intronless RNAs through an RGG motif, Genes Dev. 12 (1998), pp. 868-879.
Sarkar, N. N. Mifepristone: bioavailability, pharmacokinetics and use-effectiveness. 2002. Euro J Obst Gyneco and Repro Bio. 101:113-120.
Schuitemaker, H. et al. (1992) J. Virol. 66:1354
Sherman, M. P., de Noronha, C. M., Pearce, D., and Greene, W. C. (2000) J. Virol. 2000 74, 8159-8165.
Shi et al., 2003 J. Shi, Y. Feng, A. C. Goulet, R. R. Vaillancourt, N. A. Sachs, J. W. Hershey and M. A. Nelson, The p34cdc2-related cyclin-dependent kinase 11 interacts with the p47 subunit of eukaryotic initiation factor 3 during apoptosis, J. Biol. Chem. 278 (2003), pp. 5062-5071.
Shibata, H., Spencer, T. E., Onate, T. E., Genster, S. Y., Tsai, S. Y., Tsai, M. J., and O'Malley, B. W. (1997) Recent Prog. Horm. Res. 52, 141-164.
Shibata, R., et al. (1990a) J. Med. Primatol. 19:217-225
Shibata, R., et al. (1990b) J. Virol. 64:742-747
Sizova D V, Kolupaeva V G, Pestova T V, Shatsky I N, Hellen C U T. 1998. Specific interaction of eukaryotic translation initiation factor 3 with the 59 nontranslated regions of hepatitis C virus and classicals wine fever virus RNAs. J Virol 72:4775-4782.
Soliman et al., 1997 T. M. Soliman, R. M. Sandri-Goldin and S. J. Silverstein, Shuttling of the herpes simplex virus type 1 regulatory protein ICP27 between the nucleus and cytoplasm mediates the expression of late proteins, J. Virol. 71 (1997), pp. 9188-9197.
Sonenberg and Dever, 2003 N. Sonenberg and T. E. Dever, Eukaryotic translation initiation factors and regulators, Curr. Opin. Struct. Biol. 13 (2003), pp. 56-63.
Song et al., 2001 B. Song, K. C. Yeh, J. J. Liu and D. M. Knipe, Herpes simplex virus gene products required for viral infection of expression of G1-phase functions, Virology 290 (2001), pp. 320-328.
Swahn, M. L., G. Wang, A. R. Aedo, S. Z. Cekan and M. Bygdeman, Plasma levels of anti progestin RU486 following oral administration to non-pregnant and early pregnant women. Contraception 34 (1986), pp. 469-481.
Tang S, Collier A J, Elliott R M. 1999. Alterations to both the primary and predicted secondary structure of stem-loop IIIc of the hepatitis C virus 1b 59 untranslated region (59UTR) lead to mutants severely defective in translation which cannot be complemented in transby the wild-type 59UTR sequence. J Virol 73:2359-2364.
Taylor and Knipe, 2004 T. J Taylor and D. M. Knipe, Proteomics of herpes simplex virus replication compartments: association of cellular DNA replication, repair, recombination, and chromatin remodeling proteins with ICP8, J. Virol. 78 (2004), pp. 5856-5866.
Uprichard and Knipe, 1996 S. L. Uprichard and D. M. Knipe, Herpes simplex virus ICP27 mutant viruses exhibit reduced expression of specific DNA replication genes, J. Virol. 70 (1996), pp. 1969-1980.
Valentin, A. et al. (1991) J. AIDS 4:751
Varaklioti A, Georgopoulou U, Kakkanas A, Psaridi L, Serwe M, Casel-mann W H, Mavromara P. 1998. Mutational analysis of two un-structured domains of the 59 untranslated region of HSV RNA. Biochem Biophys Res Commun 253:678-685.
Wang C, Le S Y, Ali N, Siddiqui A. 1995. An RNA pseudoknot is an essential structural element of the internal ribosome entry site located within the hepatitis C virus 59 noncoding region. RNA1:526-537.
Wang C, Sarnow P, Siddiqui A. 1994. A conserved helical element is essential for internal initiation of translation of hepatitis C virus RNA. J Virol 68:7301-7307.
Westervelt, P. et al. (1992) J. Virol. 66:3925
Wong-Staal, F., et al (1987) AIDS Res. Hum. Retroviruses 3:33-39
Yen J H, Chang S C, Hu C R, Chu S C, Lin S S, Hsieh Y S, Chang M F. 1995. Cellular proteins specifically bind to the 59-noncoding region of hepatitis C Virus RNA. Virology 208:723-732. 206 J. S. Kieft et al.
Yu, X. F., et al. (1990) J. Virol. 64:5688-5693
Yuan, X., et al., (1990) AIDS Res. Hum Retroviruses 6:1265-1271
Zack, J. A. et al. (1988) Science 240:1026
Zhou and Knipe, 2002 C. Zhou and D. M. Knipe, Association of herpes simplex virus type 1 ICP8 and ICP27 proteins with cellular RNA polymerase II holoenzyme, J. Virol. 76 (2002), pp. 5893-5904.

### Compounds:

### D1:

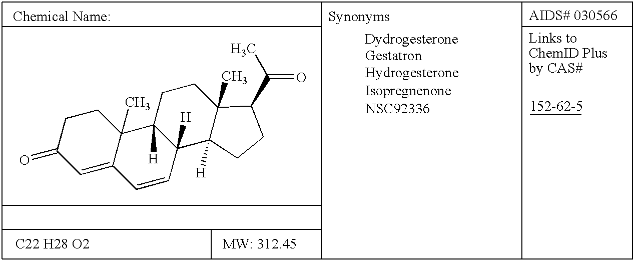

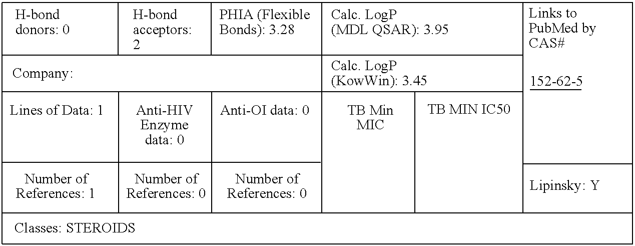

D1: Pregna-4,6-diene-3,20-dione

### Sigma Product Number: R19, 725-4.

### MDL Number: MFCD00199858.

### D2:

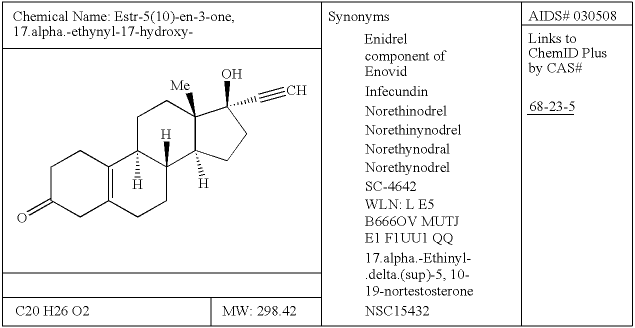

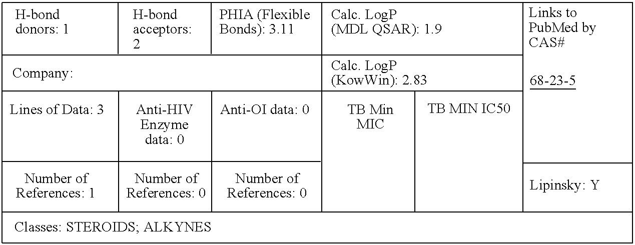

D2: 17-α-ethynyl-17-β-hydroxyestr-5 (10)-En-3-one

### Sigma Product Number: R18, 844-1.

### MDL Number: MFCD00199015.

### D3:

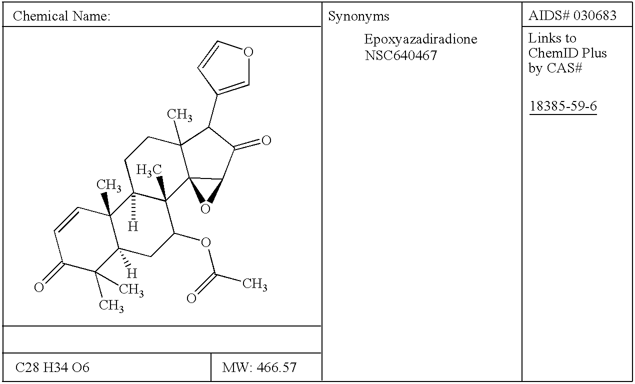

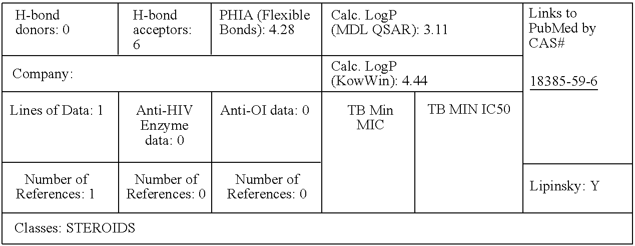

### D4:

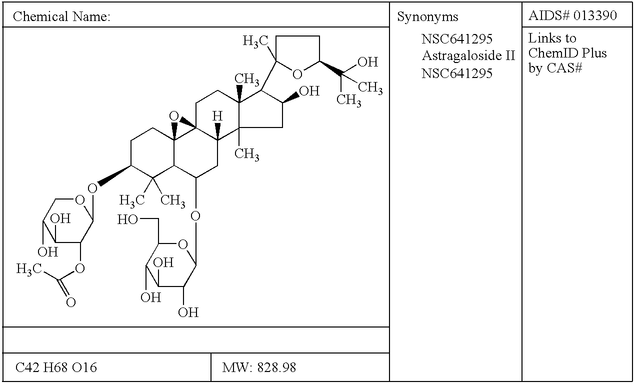

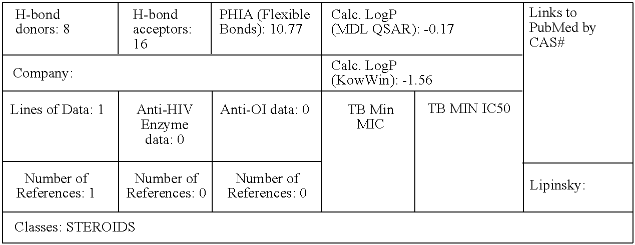

### D5:

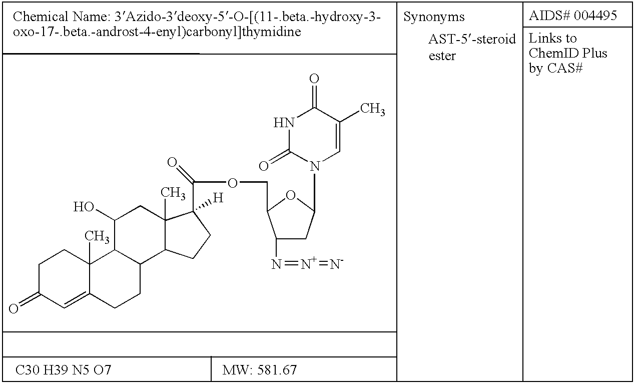

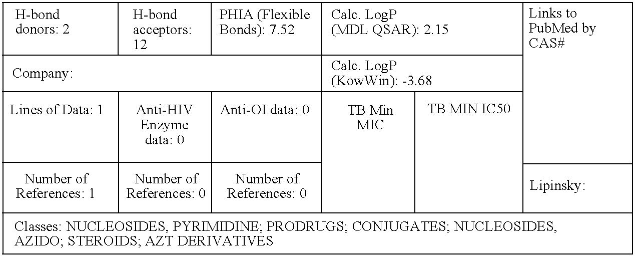

### D5: Combination of Hydrocortisone Acetate and Zidovudine

### Hydrocortisone Acetate Sigma Product Number: H4126

### Zidovudine Sigma Product Number: 11546

| | Compounds | References | |
|---|---|---|---|
| D6 | pregnenolone 16-alpha-carbonitrile | Cell 1998, 92: 73. | |
| D7 | promegestrone | J Steriod Biochem 1988, 29: 599 | |
| D8 | progesterone | J Steriod Biochem 1988, 29: 600 | Endocrinology 1980, 107: 118 |
| D9 | cortexolone | Endocrinology 1980, 107: 117 | |
| D10 | 6-beta-bromogesterone | Endocrinology 1980, 107: 119 | |
| D11 | RU43044 | PNAS 1992, 89: 3571 | |
| D12 | RU40555 | J Endcrinol. 2001, 169: 309 | |
| D13 | spironolactone | Laryngoscope 2002, 112: 298 | |
| D14 | onapristone | Biol Pharm Bull 2002, 25: 1223 | JBC 2000, 275: 17771 |
| D15 | cyproterone acetate | Mol Pharm 2003, 63: 1012 | |
| D16 | trans 4-hydroxytamoxifen | JBC 2000, 275: 17771 | |
| D17 | RTI-3022-012 | Endocrinology 1999, 140: 1449 | |
| D18 | RTI-3022-022 | Endocrinology 1999, 140: 1450 | |

| | |
|---|---|
| D19 | 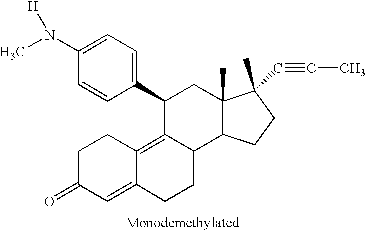 |
| D20 | 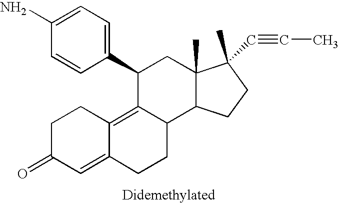 |
| D21 | 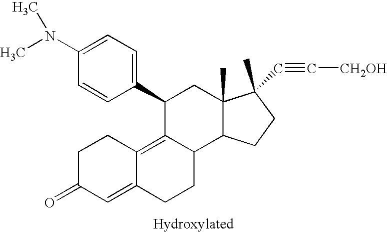 |

1. A pharmaceutical composition comprising: Polyethylene Glycol (PEG), including PEG-400 and a compound having a structure selected from the group consisting of Formulas D1-D21, and pharmaceutically acceptable salts thereof.

2. The pharmaceutical composition of claim 1 comprising Compound D1 at dosage levels effective in treating and/or preventing HIV, HCV, or HSV infection.

3. The pharmaceutical composition of claim 1 comprising Compound D2 at dosage levels effective in treating and/or preventing HIV, HCV, or HSV infection.

4. The pharmaceutical composition of claim 1 comprising Compound D3 at dosage levels effective in treating and/or preventing HIV, HCV, or HSV infection.

5. The pharmaceutical composition of claim 1 comprising Compound D4 at dosage levels effective in treating and/or preventing HIV, HCV, or HSV infection.

6. The pharmaceutical composition of claim 1 comprising Composition D5 at dosage levels effective in treating and/or preventing HIV, HCV, or HSV infection.

7. The pharmaceutical composition of claim 1 comprising Compound D6 at dosage levels effective in treating and/or preventing HIV, HCV, or HSV infection.

8. The pharmaceutical composition of claim 1 comprising Compound D7 at dosage levels effective in treating and/or preventing HIV, HCV, or HSV infection.

9. The pharmaceutical composition of claim 1 comprising Compound D8 at dosage levels effective in treating and/or preventing HIV, HCV, or HSV infection.

10. The pharmaceutical composition of claim 1 comprising Compound D9 at dosage levels effective in treating and/or preventing HIV, HCV, or HSV infection.

11. The pharmaceutical composition of claim 1 comprising Composition D10 at dosage levels effective in treating and/or preventing HIV, HCV, or HSV infection.

12. The pharmaceutical composition of claim 1 comprising Compound D11 at dosage levels effective in treating and/or preventing HIV, HCV, or HSV infection.

13. The pharmaceutical composition of claim 1 comprising Compound D12 at dosage levels effective in treating and/or preventing HIV, HCV, or HSV infection.

14. The pharmaceutical composition of claim 1 comprising Compound D13 at dosage levels effective in treating and/or preventing HIV, HCV, or HSV infection.

15. The pharmaceutical composition of claim 1 comprising Compound D14 at dosage levels effective in treating and/or preventing HIV, HCV, or HSV infection.

16. The pharmaceutical composition of claim 1 comprising Composition D15 at dosage levels effective in treating and/or preventing HIV, HCV, or HSV infection.

17. The pharmaceutical composition of claim 1 comprising Composition D16 at dosage levels effective in treating and/or preventing HIV, HCV, or HSV infection.

18. The pharmaceutical composition of claim 1 comprising Composition D17 at dosage levels effective in treating and/or preventing HIV, HCV, or HSV infection.

19. The pharmaceutical composition of claim 1 comprising Composition D18 at dosage levels effective in treating and/or preventing HIV, HCV, or HSV infection.

20. The pharmaceutical composition of claim 1 comprising Composition D19 at dosage levels effective in treating and/or preventing HIV, HCV, or HSV infection.

21. The pharmaceutical composition of claim 1 comprising Composition D20 at dosage levels effective in treating and/or preventing HIV, HCV, or HSV infection.

22. The pharmaceutical composition of claim 1 comprising Composition D21 at dosage levels effective in treating and/or preventing HIV, HCV, or HSV infection.

23. The pharmaceutical composition of claim 1 comprising: a pharmaceutically acceptable carrier or diluent; and, a compound having a structure selected from the group consisting of Formula D1-D121, and pharmaceutically acceptable salts thereof and further comprising a compound having a structure selected from the group consisting: mifepristone, zidovudine (AZT), abacavir, 3TC, d4T, ddI, ddC, efavirenz, nevirapine, delavidine, amprenavir, Indinavir, Lopinavir, nelfinavir, ritonavir, sanquinavir, acyclovir, ganciclovir, foscarnet, lamivudine, ribavirin, peginterferon interferon alpha-2a, and interferon alpha-2b, alfa-2a, and peginterferon alfa-2b

24. A method of treating an individual who is infected with HIV, HCV, or HSV comprising the step of administering to said individual a therapeutically effective amount of a composition according to claim 1.

25. A method of preventing HIV, HCV, or HSV infection in an individual identified as being a high risk individual, the method comprising the step of administering to said individual a prophylactically effective amount of a composition according to claim 1.
